(19) United States
(12) Patent Application Publication
(10) Pub. No.: US 2011/0092983 A1
(43) Pub. Date: Apr. 21, 2011

(54) MRI Biopsy Targeting Cube with Locking Flap

(76) Inventors: Ajay D. Pawar, Pune (IN); Abhijit G. Kulkarni, Pune (IN); Santosh G. Deshmukh, Pune (IN); Anil R. Jadhav, Pune (IN); Nitin P. Wale, Pune (IN)

(21) Appl. No.: 12/580,300

(22) Filed: Oct. 16, 2009

(51) Int. Cl. A61B 19/00; A61B 10/02

(52) U.S. Cl. 606/130; 600/562

(57) ABSTRACT

A biopsy system comprises a control module, a localization assembly, a biopsy device, and a targeting cube. A probe and/or other associated components of the biopsy device are configured to selectively couple with the targeting cube, which is configured to selectively couple with a grid plate. The targeting cube may comprise a locking flap for securing the targeting cube within the grid plate and/or for securing the probe and/or other associated components within the guide hole of the targeting cube. The locking flap may be operatively configured to deflect and/or compress thereby providing assistance in securing the targeting cube within the grid plate and/or securing the probe and/or other associated components within the guide hole of the targeting cube.

### BACKGROUND

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. Merely exemplary biopsy devices are disclosed in U.S. Pat. No. 6,273,862, entitled “Surgical Device for the Collection of Soft Tissue,” issued Aug. 14, 2001; U.S. Pat. No. 6,231,522, entitled “Biopsy Instrument with Breakable Sample Segments,” issued May 15, 2001; U.S. Pat. No. 6,228,055, entitled “Devices for Marking and Defining Particular Locations in Body Tissue,” issued May 8, 2001; U.S. Pat. No. 6,120,462, entitled “Control Method for an Automated Surgical Biopsy Device,” issued Sep. 19, 2000; U.S. Pat. No. 6,086,544, entitled “Control Apparatus for an Automated Surgical Biopsy Device,” issued Jul. 11, 2000; U.S. Pat. No. 6,077,230, entitled “Biopsy Instrument with Removable Extractor,” issued Jun. 20, 2000; U.S. Pat. No. 6,017,316, entitled “Vacuum Control System and Method for Automated Biopsy Device,” issued Jan. 25, 2000; U.S. Pat. No. 6,007,497, entitled “Surgical Biopsy Device,” issued Dec. 28, 1999; U.S. Pat. No. 5,980,469, entitled “Method and Apparatus for Automated Biopsy and Collection of Soft Tissue,” issued Nov. 9, 1999; U.S. Pat. No. 5,964,716, entitled “Method of Use for a Multi-Port Biopsy Instrument,” issued Oct. 12, 1999; U.S. Pat. No. 5,928,164, entitled “Apparatus for Automated Biopsy and Collection of Soft Tissue,” issued Jul. 27, 1999; U.S. Pat. No. 5,775,333, entitled “Apparatus for Automated Biopsy and Collection of Soft Tissue,” issued Jul. 7, 1998; U.S. Pat. No. 5,769,086, entitled “Control System and Method for Automated Biopsy Device,” issued Jun. 23, 1998; U.S. Pat. No. 5,649,547, entitled “Methods and Devices for Automated Biopsy and Collection of Soft Tissue,” issued Jul. 22, 1997; U.S. Pat. No. 5,526,822, entitled “Method and Apparatus for Automated Biopsy and Collection of Soft Tissue,” issued Jun. 18, 1996; U.S. Pub. No. 2008/0214955, entitled “Presentation of Biopsy Sample by Biopsy Device,” published Sep. 4, 2008; U.S. Pub. No. 2007/0255168, entitled “Grid and Rotatable Cube Guide Localization Fixture for Biopsy Device,” published Nov. 1, 2007; U.S. Pub. No. 2007/0118048, entitled “Remote Thumbwheel for a Surgical Biopsy Device,” published May 24, 2007; U.S. Pub. No. 2005/0283069, entitled “MRI Biopsy Device Localization Fixture,” published Dec. 22, 2005; U.S. Pub. No. 2003/0199753, entitled “MRI Compatible Biopsy Device with Detachable Probe,” published Oct. 23, 2003; U.S. Pub. No. 2003/0109803, entitled “MRI Compatible Surgical Biopsy Device,” published Jun. 12, 2003; U.S. Pub. No. 2008/0221480, entitled “Biopsy Sample Storage,” published Sep. 11, 2008; and U.S. Pub. No. 2008/0146962, entitled “Biopsy System with Vacuum Control Module,” published Jun. 19, 2008. The disclosure of each of the above-cited U.S. Patents and U.S. Patent Application Publications is incorporated by reference herein.

Some biopsy systems may provide an apparatus to guide a probe and/or other components of a biopsy device to a desired biopsy site. In some such biopsy systems, a guide cube and positioning grid plate may be used. The guide cube may be selectively located within an opening in the grid plate. The guide cube may include guide holes to receive a portion of the probe and/or other components, for example a needle, cannula, obturator, or combinations of these or other components. With the guide cube inserted in the grid plate, the probe or other components can be guided through a selected guide hole of the guide cube to arrive at a desired biopsy site. The desired biopsy site may or may not have been identified and/or targeted by one or more of the guidance approaches mentioned above. In some situations, it might be desirable to provide a guide cube with features that improve a guide cube's use with one or more positioning grid plates. Merely exemplary biopsy device guides are disclosed in U.S. patent application Ser. No. 12/485,119, entitled “Biopsy Targeting Cube with Elastomeric Edges,” filed Jun. 16, 2009; U.S. patent application Ser. No. 12/485,138, entitled “Biopsy Targeting Cube with Elastomeric Body,” filed Jun. 16, 2009; U.S. patent application Ser. No. 12/485,168, entitled “Biopsy Targeting Cube with Malleable Members,” filed Jun. 16, 2009; U.S. patent application Ser. No. 12/485,278, entitled “Biopsy Targeting Cube with Angled Interface,” filed June 16, 2009; and U.S. patent application Ser. No. 12/485,318, entitled “Biopsy Targeting Cube with Living Hinges,” filed June 16, 2009. The disclosure of each of the above-cited U.S. Patent Applications is incorporated by reference herein.

While several systems and methods have been made and used for obtaining a biopsy sample, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings. In the drawings, like numerals represent like elements throughout the several views.

FIG. 1 is a perspective view of a biopsy system including a control module remotely coupled to a biopsy device, and including a localization assembly.

FIG. 2 is a perspective view of a breast coil of the localization assembly of FIG. 1.

FIG. 3 is a perspective view of the biopsy device inserted through the guide cube of the localization assembly of FIG. 1.

FIG. 4 is a perspective view of the obturator and cannula of the biopsy system of FIG. 1.

FIG. 5 is an exploded perspective view of the obturator and cannula of FIG. 4.

FIG. 6 is a perspective view of the guide cube inserted into the grid plate of the localization assembly of FIG. 1.

FIG. 7 is a perspective view of the obturator and cannula of FIG. 4 with a depth stop device of FIG. 1 inserted through the guide cube and grid plate of FIG. 6.

FIG. 8 is a perspective view of the guide cube of the biopsy system of FIG. 1.

FIG. 9 is a diagram of nine guide positions achievable by rotating the guide cube of FIG. 8.

FIG. 10 is a perspective view of another guide cube for the biopsy system of FIG. 1 with a self-grounding feature.

FIG. 11 is a perspective view of the obturator and cannula of FIG. 1 inserted into one of two guide cubes of FIG. 10 inserted into the grid plate of FIG. 1.

FIG. 12 is a perspective view of another guide cube having an open top and bottom with another self-grounding feature.

FIG. 13 is a rear perspective view of another guide cube with another self-grounding feature.

FIG. 14 is a front perspective view of the guide cube of FIG. 13.

FIG. 15 is a right side view of the guide cube of FIG. 13 with angled, parallel guide holes depicted in phantom.

FIG. 16 is a front perspective view of an exemplary guide cube with a locking flap.

FIG. 17 is a front perspective view of another exemplary guide cube with a locking flap.

FIG. 18 is a front perspective view of the locking flap of FIG. 16.

FIG. 19 is a front perspective view of the guide cube of FIG. 16 without the locking flap.

FIG. 20 is a front perspective view of the locking flap of FIG. 17.

FIG. 21 is a front perspective view of the guide cube of FIG. 17 without the locking flap.

FIG. 22 is a front perspective view of another locking flap having overlapping guide holes.

FIG. 23 is a front elevational view of the guide cube of FIG. 16, with a portion of the locking flap being deflected away from a corresponding face of the guide cube.

FIG. 24 is a top plan view of the guide cube of FIG. 23.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples should not be used to limit the scope of the present invention. Other features, aspects, and advantages of the versions disclosed herein will become apparent to those skilled in the art from the following description, which is by way of illustration, one of the best modes contemplated for carrying out the invention. As will be realized, the versions described herein are capable of other different and obvious aspects, all without departing from the invention. Accordingly, the drawings and descriptions should be regarded as illustrative in nature and not restrictive.

As shown in the figures, an exemplary magnetic resonance imaging (MRI or MR imaging) compatible biopsy system may include a control module (12), localization assembly (15), and biopsy device (14). In particular, localization assembly (15) is configured to localize a patient's breast and guide needle (90) of biopsy device (14) to a targeted area within the patient's breast; while control module (12) is operable to control biopsy device (14) after needle (90) has been introduced to the target site. These components and their sub-components will be discussed further below. In addition, guide cubes for use with various localization assemblies will be discussed. While this disclosure may reference the biopsy system as compatible with MRI and MM equipment and devices, it should be appreciated that other imaging techniques and equipment and devices may be used with the components described below, including but not limited to stereotactic, ultrasound, PEM, BSGI, and/or other imaging techniques and equipment.

I. Control Module

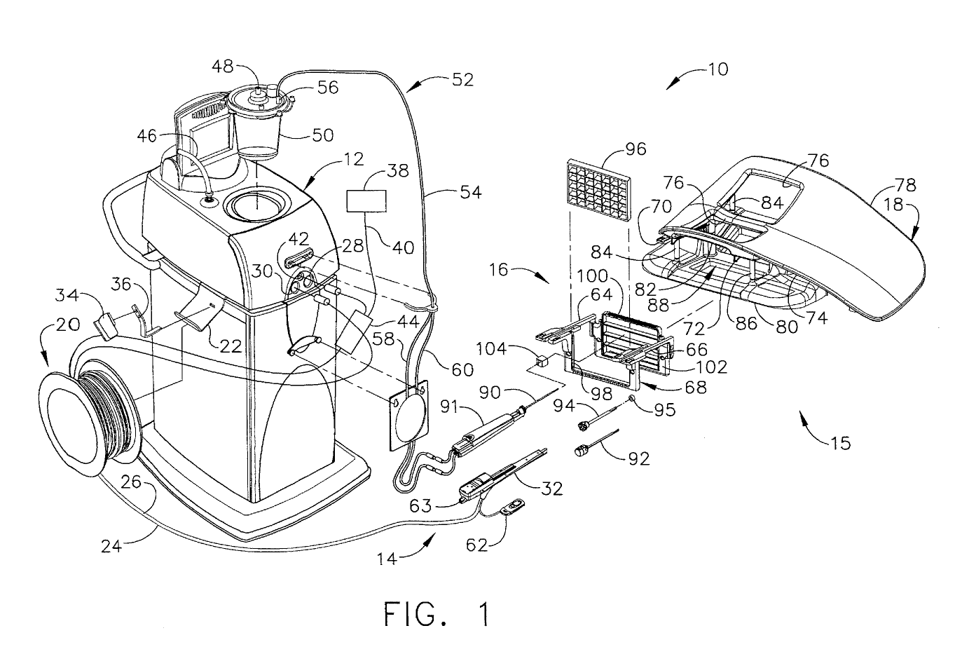

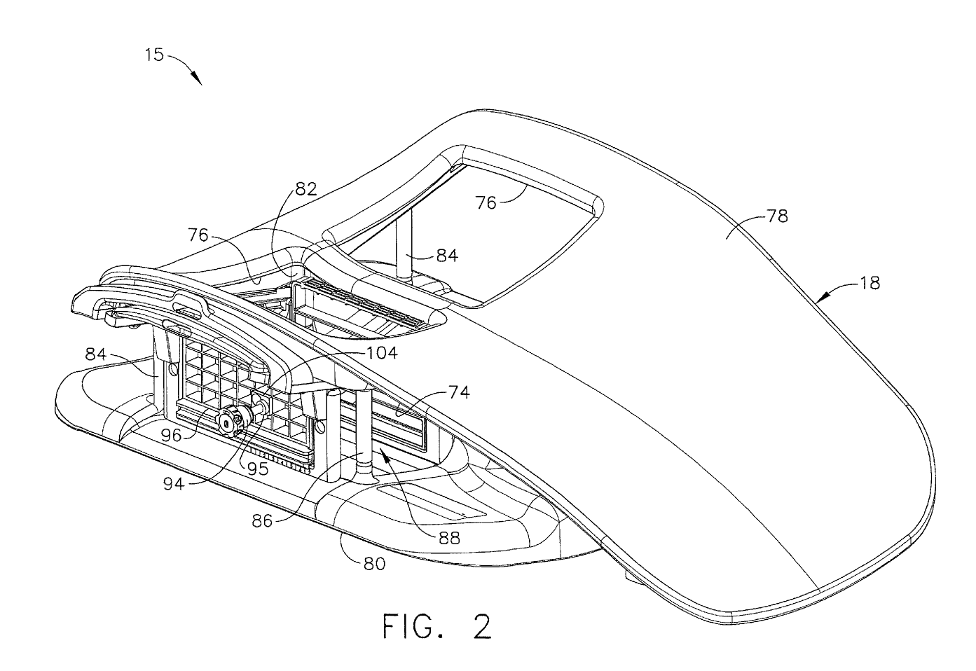

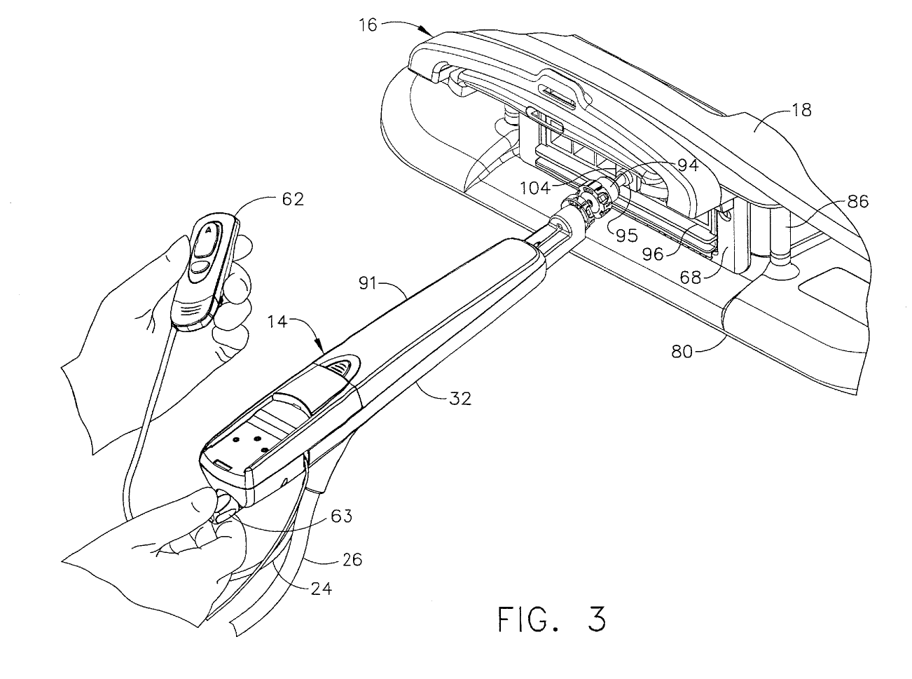

In FIGS. 1-3, MRI compatible biopsy system (10) has control module (12) that may be placed outside of a shielded room containing an MM machine (not shown) or at least spaced away to mitigate detrimental interaction with its strong magnetic field and/or sensitive radio frequency (RF) signal detection antennas. As described in U.S. Pat. No. 6,752,768, which is hereby incorporated by reference in its entirety, a range of preprogrammed functionality may be incorporated into control module (12) to assist in taking tissue samples. Control module (12) controls and powers biopsy device (14) that is used with localization assembly (15). Biopsy device (14) is positioned and guided by localization fixture (16) attached to breast coil (18) that may be placed upon a gantry (not shown) of a MRI or other imaging machine.

In the present example, control module (12) is mechanically, electrically, and pneumatically coupled to biopsy device (14) so that components may be segregated that need to be spaced away from the strong magnetic field and the sensitive RF receiving components of a MRI machine. Cable management spool (20) is placed upon cable management attachment saddle (22) that projects from a side of control module (12). Wound upon cable management spool (20) is paired electrical cable (24) and mechanical cable (26) for communicating control signals and cutter rotation/advancement motions respectively. In particular, electrical and mechanical cables (24, 26) each have one end connected to respective electrical and mechanical ports (28, 30) in control module (12) and another end connected to holster portion (32) of biopsy device (14). Docking cup (34), which may hold holster portion (32) when not in use, is hooked to control module (12) by docking station mounting bracket (36). It should be understood that such components described above as being associated with control module (12) are merely optional.

Interface lock box (38) mounted to a wall provides tether (40) to lockout port (42) on control module (12). Tether (40) is uniquely terminated and of short length to preclude inadvertent positioning of control module (12) too close to a MRI machine or other machine. In-line enclosure (44) may register tether (40), electrical cable (24) and mechanical cable (26) to their respective ports (42, 28, 30) on control module (12).

Vacuum assist is provided by first vacuum line (46) that connects between control module (12) and outlet port (48) of vacuum canister (50) that catches liquid and solid debris. Tubing kit (52) completes the pneumatic communication between control module (12) and biopsy device (14). In particular, second vacuum line (54) is connected to inlet port (56) of vacuum canister (50). Second vacuum line (54) divides into two vacuum lines (58, 60) that are attached to biopsy device (14). With biopsy device (14) installed in holster portion (32), control module (12) performs a functional check. Saline may be manually injected into biopsy device (14) or otherwise introduced to biopsy device (14), such as to serve as a lubricant and to assist in achieving a vacuum seal and/or for other purposes. Control module (12) actuates a cutter mechanism (not shown) in biopsy device (14), monitoring full travel of a cutter in biopsy device (14) in the present example. Binding in mechanical cable (26) or within biopsy device (14) may optionally monitored with reference to motor force exerted to turn mechanical cable (26) and/or an amount of twist in mechanical cable (26) sensed in comparing rotary speed or position at each end of mechanical cable (26).

Remote keypad (62), which is detachable from holster portion (32), communicates via electrical cable (24) to control panel (12) to enhance clinician control of biopsy device (14) in the present example, especially when controls that would otherwise be on biopsy device (14) itself are not readily accessible after insertion into localization fixture (16) and/or placement of control module (12) is inconveniently remote (e.g., 30 feet away). However, as with other components described herein, remote keypad (62) is merely optional, and may be modified, substituted, supplemented, or omitted as desired. In the present example, aft end thumbwheel (63) on holster portion (32) is also readily accessible after insertion to rotate the side from which a tissue sample is to be taken.

Of course, the above-described control module (12) is merely one example. Any other suitable type of control module (12) and associated components may be used. By way of example only, control module (12) may instead be configured and operable in accordance with the teachings of U.S. Pub. No. 2008/0228103, entitled “Vacuum Timing Algorithm for Biopsy Device,” published Sep. 18, 2008, the disclosure of which is incorporated by reference herein. As another merely illustrative example, control module (12) may instead be configured and operable in accordance with the teachings of U.S. patent application Ser. No. 12/337,814, entitled “Control Module Interface for MRI Biopsy Device,” filed Dec. 18, 2008, the disclosure of which is incorporated by reference herein. Alternatively, control module (12) may have any other suitable components, features, configurations, functionalities, operability, etc. Other suitable variations of control module (12) and associated components will be apparent to those of ordinary skill in the art in view of the teachings herein.

II. Localization Assembly

Localization assembly (15) of the present example comprises breast coil (18) and localization fixture (16). These components of localization assembly (15) are described further below.

Left and right parallel upper guides (64, 66) of localization framework (68) are laterally adjustably received respectively within left and right parallel upper tracks (70, 72) attached to under side (74) and to each side of a selected breast aperture (76) formed in patient support platform (78) of breast coil (18). Base (80) of breast coil (18) is connected by centerline pillars (82) that are attached to patient support platform (78) between breast apertures (76). Also, a pair of outer vertical support pillars (84, 86) on each side spaced about a respective breast aperture (76) respectively define lateral recess (88) within which localization fixture (16) resides.

It should be appreciated that the patient's breasts hang pendulously respectively into breast apertures (76) within lateral recesses (88) in the present example. For convenience, herein a convention is used for locating a suspicious lesion by Cartesian coordinates within breast tissue referenced to localization fixture (16) and to thereafter selectively position an instrument, such as needle (90) of probe (91) that is engaged to holster portion (32) to form biopsy device (14). Of course, any other type of coordinate system or targeting techniques may be used. To enhance hands-off use of biopsy system (10), especially for repeated re-imaging within the narrow confines of a closed bore MRI machine, biopsy system (10) may also guide obturator (92) encompassed by cannula (94). Depth of insertion is controlled by depth stop device (95) longitudinally positioned on either needle (90) or cannula (94). Alternatively, depth of insertion may be controlled in any other suitable fashion.

This guidance is specifically provided by a lateral fence in the present example, depicted as grid plate (96), which is received within laterally adjustable outer three-sided plate bracket (98) attached below left and right parallel upper guides (64, 66). Similarly, a medial fence with respect to a medial plane of the chest of the patient, depicted as medial plate (100), is received within inner three-sided plate bracket (102) attached below left and right parallel upper guides (64, 66) close to centerline pillars (82) when installed in breast coil (18). To further refine the insertion point of the instrument (e.g., needle (90) of probe (91), obturator/cannula (92, 94), etc.), guide cube (104) may be inserted into grid plate (96).

In the present example, the selected breast is compressed along an inner (medial) side by medial plate (100) and on an outer (lateral) side of the breast by grid plate (96), the latter defining an X-Y plane. The X-axis is vertical (sagittal) with respect to a standing patient and corresponds to a left-to-right axis as viewed by a clinician facing the externally exposed portion of localization fixture (16). Perpendicular to this X-Y plane extending toward the medial side of the breast is the Z-axis, which typically corresponds to the orientation and depth of insertion of needle (90) or obturator/cannula (92, 94) of biopsy device (14). For clarity, the term Z-axis may be used interchangeably with “axis of penetration”, although the latter may or may not be orthogonal to the spatial coordinates used to locate an insertion point on the patient. Versions of localization fixture (16) described herein allow a non-orthogonal axis of penetration to the X-Y axis to a lesion at a convenient or clinically beneficial angle.

It should be understood that the above-described localization assembly (15) is merely one example. Any other suitable type of localization assembly (15) may be used, including but not limited to localization assemblies (15) that use a breast coil (18) and/or localization fixture (16) different from those described above. Other suitable components, features, configurations, functionalities, operability, etc. for a localization assembly (15) will be apparent to those of ordinary skill in the art in view of the teachings herein.

III. Biopsy Device

As shown in FIG. 1, one version of biopsy device (14) may comprise holster portion (32) and probe (91). Exemplary holster portion (32) was discussed previously in the above section addressing control module (12). The following paragraphs will discuss probe (91) and associated components and devices in further detail.

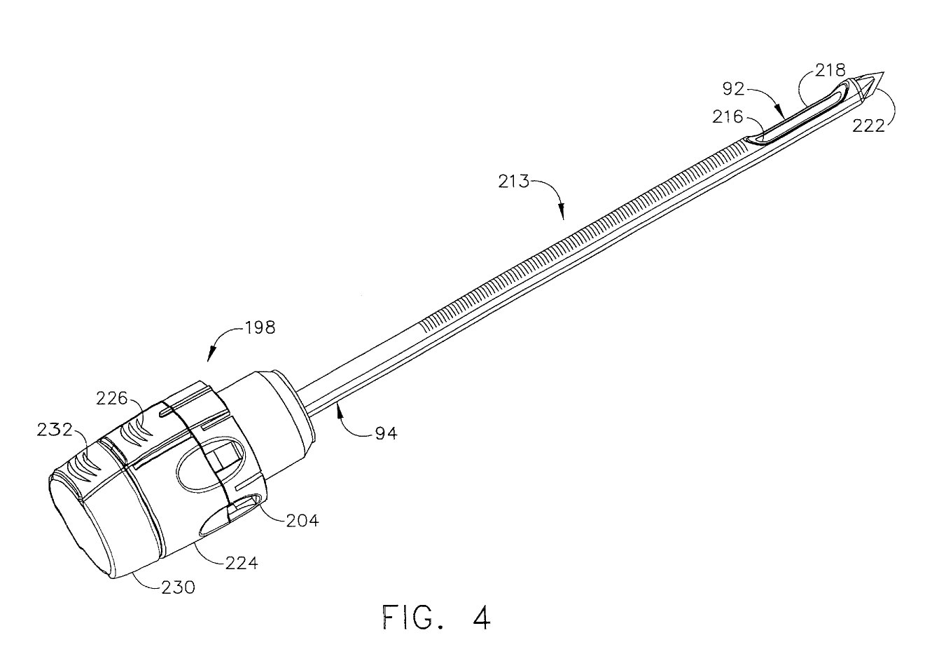

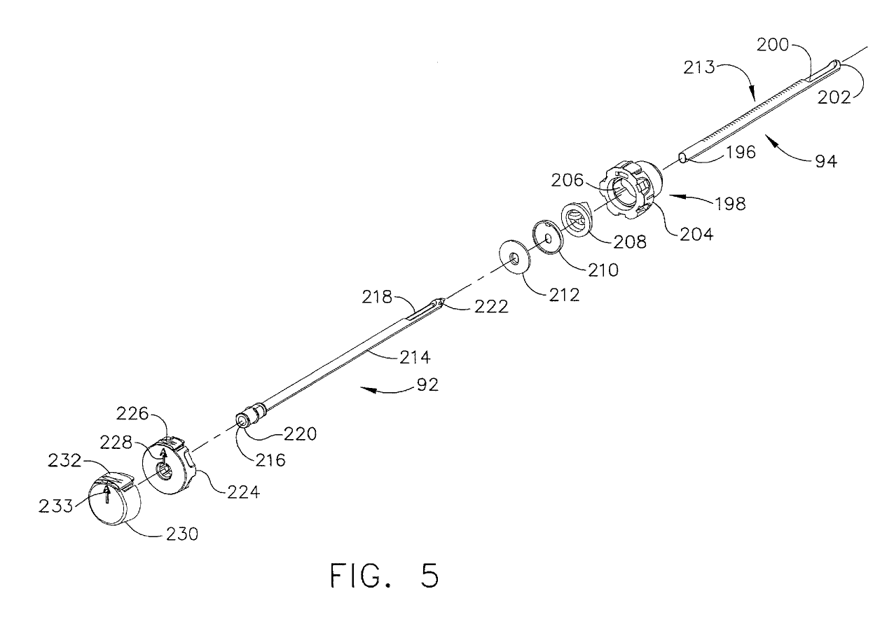

In the present example, cannula (94) and obturator (92) are associated with probe (91). In particular, and as shown in FIGS. 4, 5, and 7, obturator (92) is slid into cannula (94) and the combination is guided through guide cube (104) to the biopsy site within the breast tissue. Obturator (92) is then withdrawn from cannula (94), then needle (90) of probe (91) is inserted in cannula (94), and then biopsy device (14) is operated to acquire one or more tissue samples from the breast via needle (90).

Cannula (94) of the present example is proximally attached to cylindrical hub (198) and cannula (94) includes lumen (196) and lateral aperture (200) proximate to open distal end (202). Cylindrical hub (198) has exteriorly presented thumbwheel (204) for rotating lateral aperture (200). Cylindrical hub (198) has interior recess (206) that encompasses duckbill seal (208), wiper seal (210) and seal retainer (212) to provide a fluid seal when lumen (196) is empty and for sealing to inserted obturator (92). Longitudinally spaced measurement indicia (213) along an outer surface of cannula (94) visually, and perhaps physically, provide a means to locate depth stop device (95) of FIG. 1.

Obturator (92) of the present example incorporates a number of components with corresponding features. Hollow shaft (214) includes fluid lumen (216) that communicates between imageable side notch (218) and proximal port (220). Hollow shaft (214) is longitudinally sized to extend, when fully engaged with cannula (94), piercing tip (222) out of distal end (202) of cannula (94). Obturator thumbwheel cap (224) encompasses proximal port (220) and includes locking feature (226), which includes visible angle indicator (228), that engages cannula thumbwheel (204) to ensure that imageable side notch (218) is registered to lateral aperture (200) in cannula (94). Obturator seal cap (230) may be engaged proximally into obturator thumbwheel cap (224) to close fluid lumen (216). Obturator seal cap (230) of the present example includes locking or locating feature (232) that includes visible angle indicator (233) that corresponds with visible angle indicator (228) on obturator thumbwheel cap (224), which may be fashioned from either a rigid, soft, or elastomeric material. In FIG. 7, guide cube (104) has guided obturator (92) and cannula (94) through grid plate (96).

While obturator (92) of the present example is hollow, it should be understood that obturator (92) may alternatively have a substantially solid interior, such that obturator (92) does not define an interior lumen. In addition, obturator (92) may lack side notch (218) in some versions. Other suitable components, features, configurations, functionalities, operability, etc. for an obturator (92) will be apparent to those of ordinary skill in the art in view of the teachings herein. Likewise, cannula (94) may be varied in a number of ways. For instance, in some other versions, cannula (94) has a closed distal end (202). As another merely illustrative example, cannula (94) may have a closed piercing tip (222) instead of obturator (92) having piercing tip (222). In some such versions, obturator (92) may simply have a blunt distal end; or the distal end of obturator (92) may have any other suitable structures, features, or configurations. Other suitable components, features, configurations, functionalities, operability, etc. for a cannula (94) will be apparent to those of ordinary skill in the art in view of the teachings herein. Furthermore, in some versions, one or both of obturator (92) or cannula (94) may be omitted altogether. For instance, needle (90) of probe (91) may be directly inserted into a guide cube (104), without being inserted into guide cube (104) via cannula (94).

Another component that may be used with probe (91) (or needle (90)) is depth stop (95). Depth stop may be of any suitable configuration that is operable to prevent cannula (94) and obturator (92) (or needle (90)) from being inserted further than desired. For instance, depth stop (95) may be positioned on the exterior of cannula (94) (or needle (90)), and may be configured to restrict the extent to which cannula (94) is inserted into a guide cube. It should be understood that such restriction by depth stop (95) may further provide a limit on the depth to which the combination of cannula (94) and obturator (92) (or needle (90)) may be inserted into the patient's breast. Furthermore, it should be understood that such restriction may establish the depth within the patient's breast at which biopsy device (14) acquires one or more tissue samples after obturator (92) has been withdrawn from cannula (94) and needle (90) has been inserted in cannula (94). Exemplary depth stops (95) that may be used with biopsy system (10) are described in U.S. Pub. No. 2007/0255168, entitled “Grid and Rotatable Cube Guide Localization Fixture for Biopsy Device,” published Nov. 1, 2007, and incorporated by reference herein as mentioned previously.

In the present example, and as noted above, biopsy device (14) includes a needle (90) that may be inserted into cannula (94) after the combination of cannula (94) and obturator (92) has been inserted to a desired location within a patient's breast and after obturator (92) has been removed from cannula (94). Needle (90) of the present example comprises a lateral aperture (not shown) that is configured to substantially align with lateral aperture (200) of cannula (94) when needle (90) is inserted into lumen (196) of cannula (94). Probe (91) of the present example further comprises a rotating and translating cutter (not shown), which is driven by components in holster (32), and which is operable to sever tissue protruding through lateral aperture (200) of cannula (94) and the lateral aperture of needle (90). Severed tissue samples may be retrieved from biopsy device (14) in any suitable fashion.

By way of example only, biopsy device (14) may be configured and operable in accordance with the teachings of U.S. Pub. No. 2008/0228103, entitled “Vacuum Timing Algorithm For Biopsy Device,” published Sep. 18, 2008, the disclosure of which is incorporated by reference herein. As another merely illustrative example, biopsy device (14) may be configured and operable in accordance with the teachings of U.S. patent application Ser. No. 12/337,874, entitled “Mechanical Tissue Sample Holder Indexing Device,” filed Dec. 18, 2008, the disclosure of which is incorporated by reference herein. As another merely illustrative example, biopsy device (14) may be configured and operable in accordance with the teachings of U.S. patent application Ser. No. 12/337,674, entitled “Biopsy Device with Sliding Cutter Cover,” filed Dec. 18, 2008, the disclosure of which is incorporated by reference herein. By way of example only, cannula (94) may be replaced with any of the detachable needles described in U.S. patent application Ser. No. 12/337,674, entitled “Biopsy Device with Sliding Cutter Cover.” As another merely illustrative example, biopsy device (14) may be configured and operable in accordance with the teachings of U.S. Patent Application Serial No. 12/337,911, entitled “Biopsy Device with Discrete Tissue Chambers,” filed Dec. 18, 2008, the disclosure of which is incorporated by reference herein. As another merely illustrative example, biopsy device (14) may be configured and operable in accordance with the teachings of U.S. patent application Ser. No. 12/337,942, entitled “Biopsy Device with Central Thumbwheel,” filed Dec. 18, 2008, the disclosure of which is incorporated by reference herein. Alternatively, biopsy device (14) may have any other suitable components, features, configurations, functionalities, operability, etc. Other suitable variations of biopsy device (14) and associated components will be apparent to those of ordinary skill in the art in view of the teachings herein

IV. Guide Cubes

Guide cubes described below are generally adapted for use with a localization assembly (15) as described above. Numerous features of merely exemplary guide cubes will be discussed in the paragraphs that follow.

A. Guide Cubes Generally

In some versions, guide cubes may comprise a body defined by one or more edges and faces. The body may include one or more guide holes or other types of passages that extend between faces of the guide cube and that may be used to guide an instrument such as a biopsy device (14) or a portion of a biopsy device (14) (e.g., needle (90) of biopsy device (14), a combination of cannula (94) and obturator (92), etc.). Guide cubes may be rotatable about one, two, or three axes to position the one or more guide holes or passages of the guide cube into a desired position.

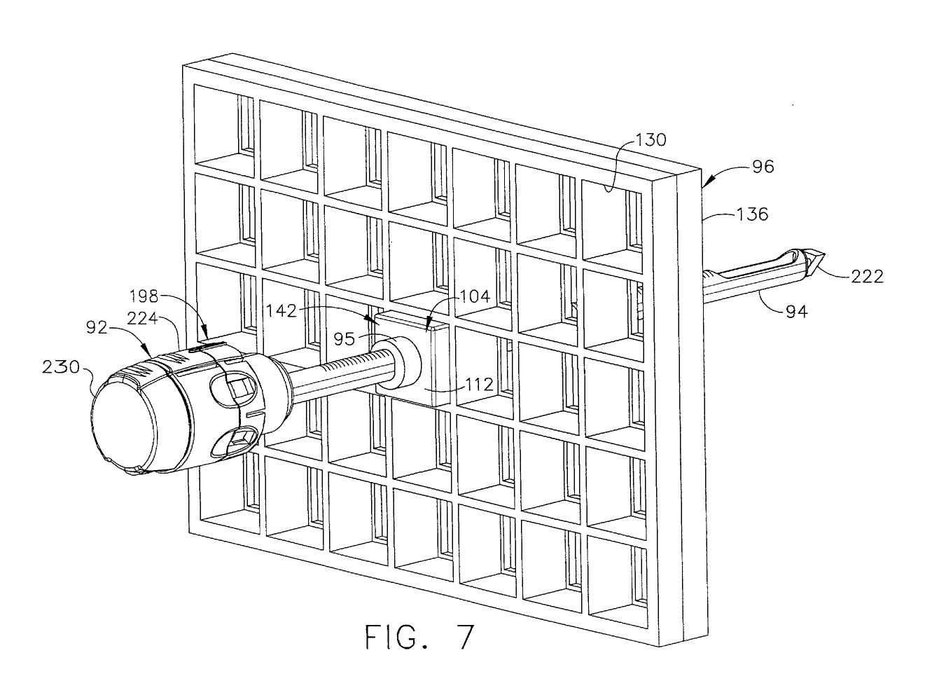

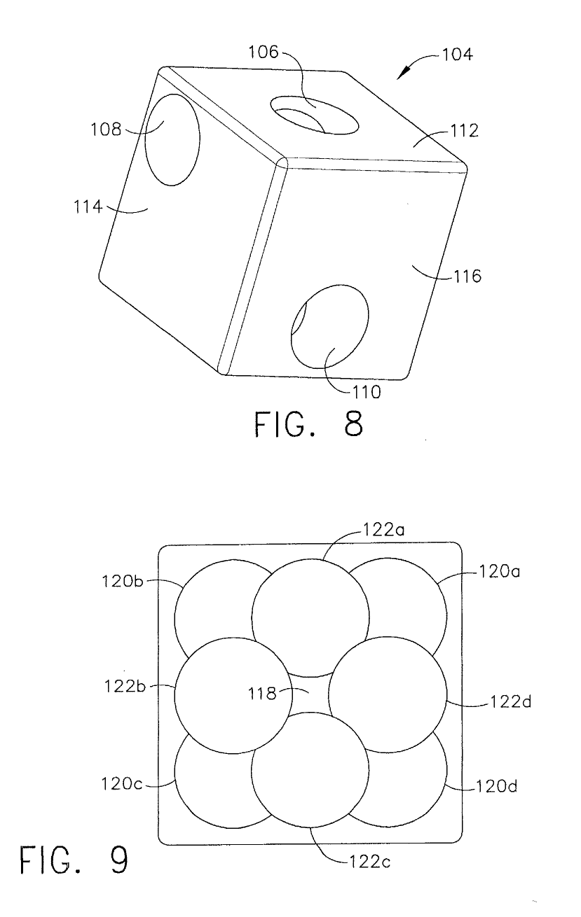

Referring now to FIG. 8, guide cube (104), includes central guide hole (106), corner guide hole (108), and off-center guide hole (110) that pass orthogonally to one another between respective opposite pairs of faces (112, 114, 116). By selectively rotating guide cube (104) in two axes, one pair of faces (112, 114, 116) may be proximally aligned to an unturned position and then the selected proximal face (112, 114, 116) optionally rotated a quarter turn, half turn, or three-quarter turn. Thereby, one of nine guide positions (118, 120a-120d, 122a-122d) may be proximally exposed as depicted in FIG. 9. More specifically, central guide hole (106) may provide for guide position (118), corner guide hole (108) may provide for guide positions (120a-120d), and off-center guide hole (110) may provide for guide positions (122a-122d).

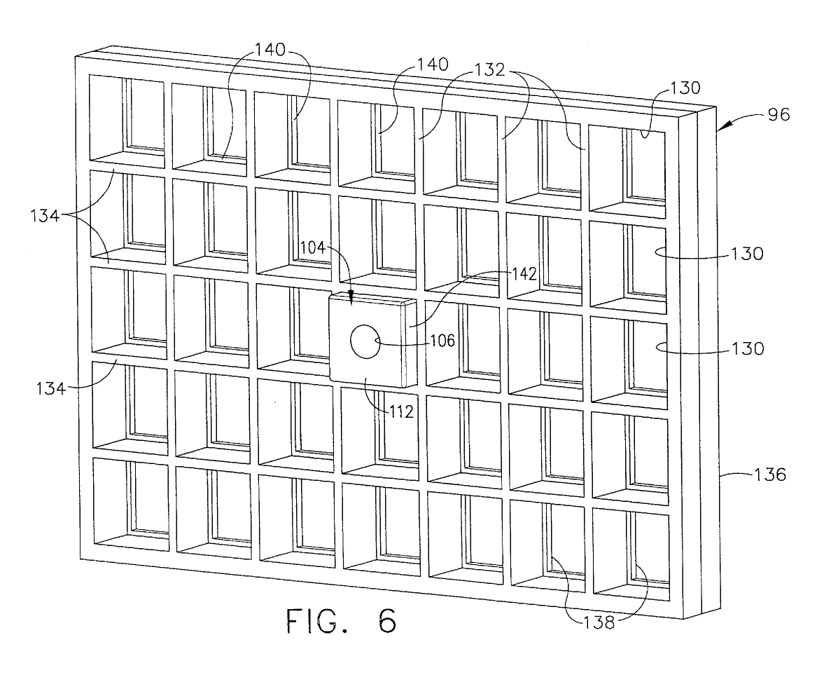

In FIG. 6, two-axis rotatable guide cube (104) is sized for insertion from a proximal side into one of a plurality of square recesses (130) in grid plate (96), which are formed by intersecting vertical bars (132) and horizontal bars (134). Guide cube (104) is prevented from passing through grid plate (96) by backing substrate (136) attached to a front face of grid plate (96). Backing substrate (136) includes respective square opening (138) centered within each square recess (130), forming lip (140) sufficient to capture the front face of guide cube (104), but not so large as to obstruct guide holes (104, 106, 108). The depth of square recesses (130) is less than guide cube (104), thereby exposing a proximal portion (142) of guide cube (104) for seizing and extraction from grid plate (96). It will be appreciated by those of ordinary skill in the art based on the teachings herein that backing substrate (136) of grid plate (96) may be omitted altogether in some versions. In some such versions without backing substrate (136) other features of a guide cube, as will be discussed in more detail below, may be used to securely and removably fit a guide cube within a grid plate. However, such other features may also be used in combination with a grid plate having backing substrate (136), such as grid plate (96), instead of partially or wholly omitting backing substrate (136).

B. Self-Grounding Guide Cubes

In FIG. 10, guide cube (104a) has self-grounding by means of added rectangular prism (240) which has a shared edge with cubic portion (242) of guide cube (104a). When viewed orthogonally to the shared cube edge, larger square face (244) of cubic portion (242) overlaps with smaller square face (246) of rectangular prism (240). As shown in FIG. 11, rectangular prism (240) allows proximal exposure of one of two adjacent faces (250, 252) of guide cube (104a) and then turning each to one of four quarter-turn rotational positions. In the illustrative version, first face (250) has central guide hole (106a) and second face (252) has corner guide hole (108a), and off-center guide hole (110a). Radial recess (254) is formed in rectangular prism (240) to allow grounding of depth stop device (95) against face (252) when off-center guide hole (110a) is used.

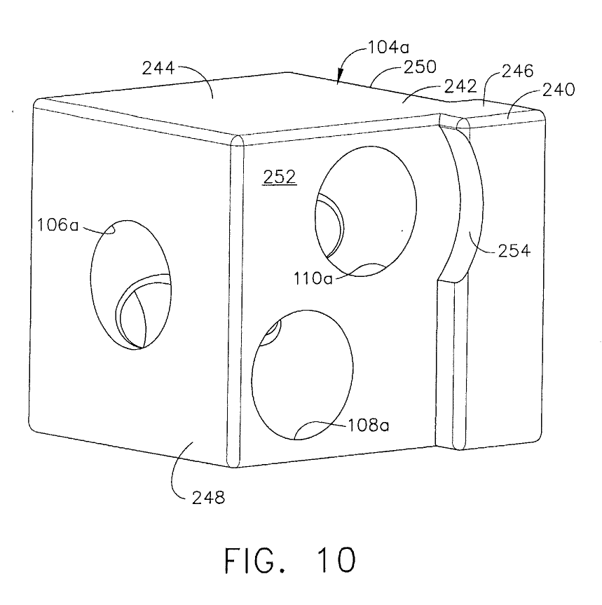

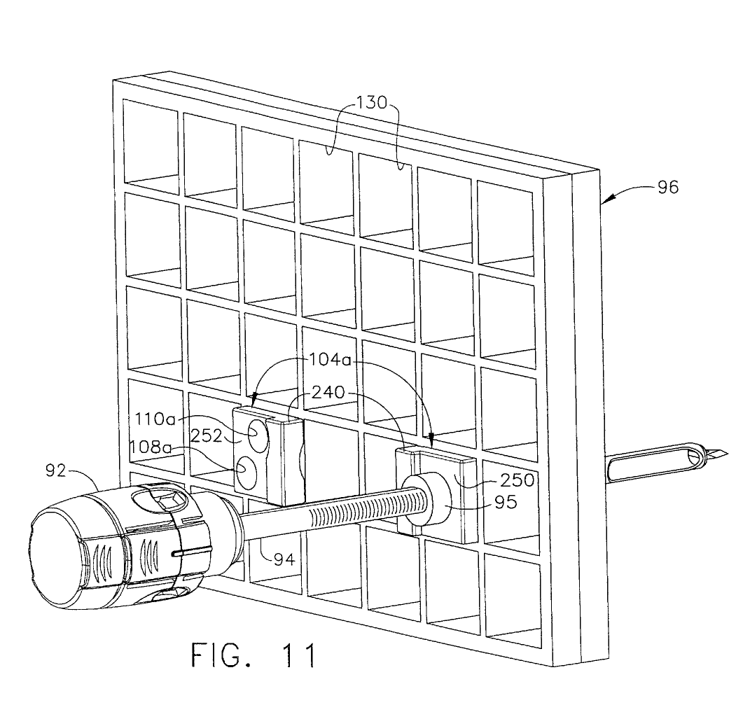

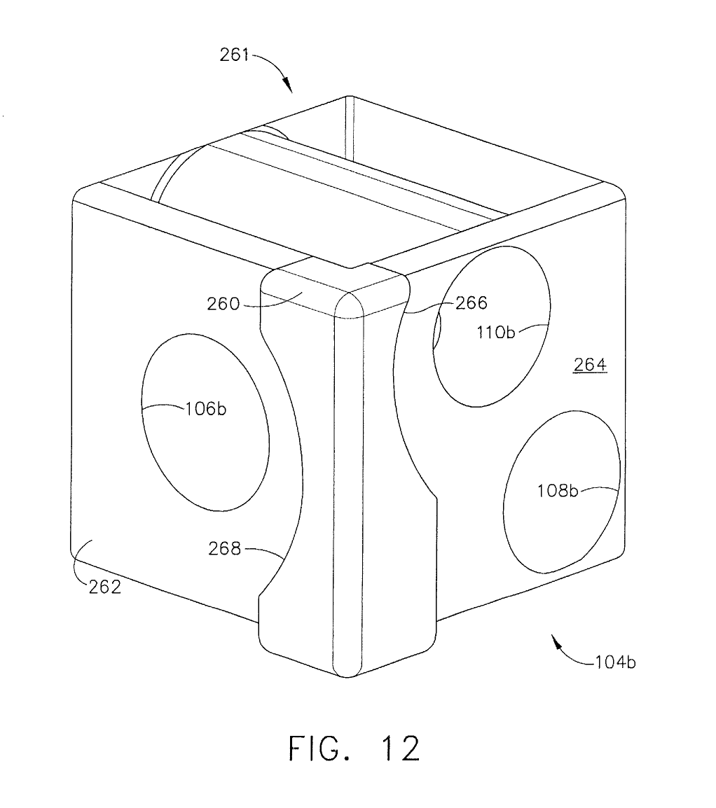

In FIG. 12, guide cube (104b) has self-grounding by means of added rectangular prism (260) that protrudes from two faces (262, 264) of guide cube (104b). Rectangular prism (260) allows proximal exposure of one of two adjacent faces (262, 264) of guide cube (104b) and then turning each to one of four quarter-turn rotational positions. In the illustrative version, first face (262) has central guide hole (106b) and second face (264) has corner guide hole (108b) and off-center guide hole (110b). First radial recess (266) is formed in rectangular prism (260) to allow grounding of depth stop device (95) against face (264) when off-center guide hole (110b) is used. Second radial recess (268) is formed in rectangular prism (260) to allow grounding of depth stop device (95) against face (262) when central guide hole (106b) is used. As discussed in greater detail below, guide cube (104b) may have open top (261) and/or an open bottom (not shown) defined by the faces of guide cube (104b) as depicted in the illustrated version.

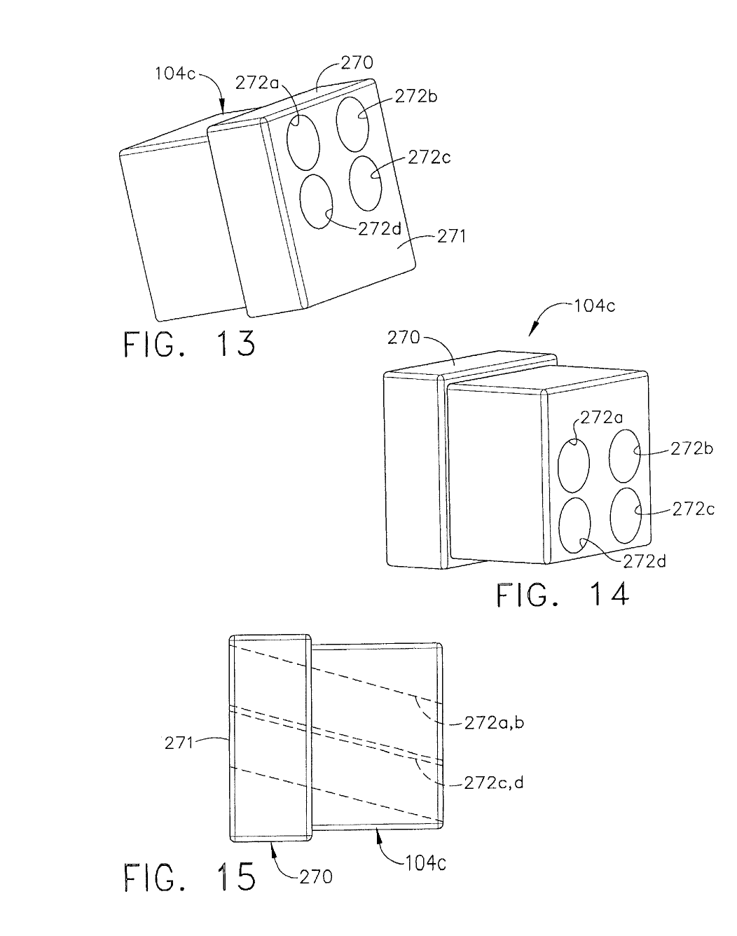

In FIGS. 13-15, guide cube (104c) has proximal enlarged hat portion (270) about proximal face (271) that grounds against selected square recess (130), such as in grid plate (96), and allows rotation about one axis to one of four quarter-turn positions. Four angled guide holes (272a, 272b, 272c, 272d) allow accessing not only an increased number of insertion points within selected square recess (130) but also a desired angle of penetration rather than being constrained to a perpendicular insertion. It will be appreciated based on the teachings herein that while angled guide holes may be used in some versions, orthogonal guide holes may be used instead of or in addition to angled guide holes in other versions.

C. Locking Flap

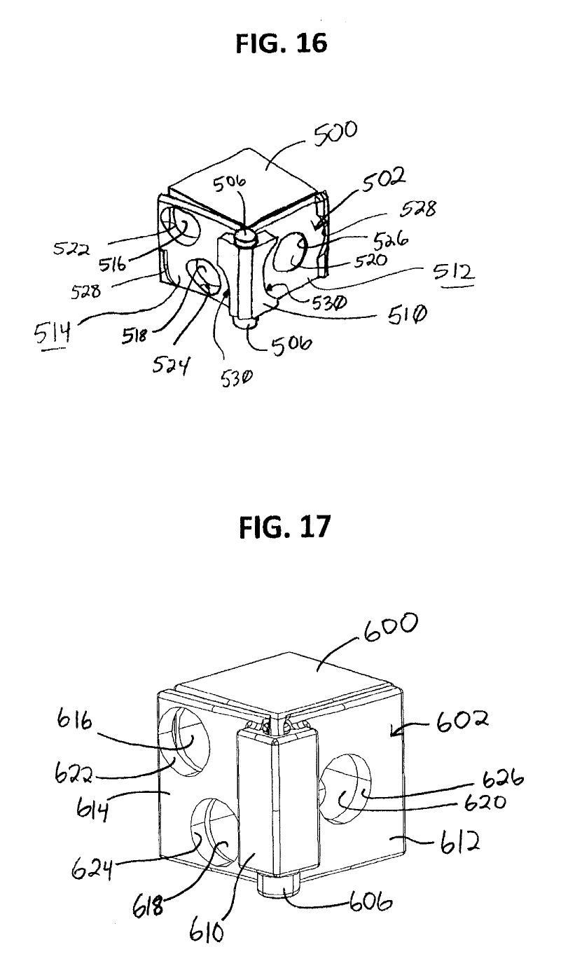

Referring now to FIGS. 16 and 17, other guide cubes (500, 600) are shown that include locking flaps (502, 602). Locking flaps (502, 602), as described further below and in the drawings, may be designed for use with other types of guide cubes, including but not limited to those described above. Alternatively, as discussed in more detail below, guide cubes (500, 600) may incorporate other features to engage with locking flaps (502, 602). It should further be appreciated that locking flaps (502, 602) may be provided as part of guide cube (500, 600), part of biopsy device (14), or part of an accessory to either or both guide cube (500, 600) and biopsy device (14).

Referring to FIGS. 16, 18-19, and 23-24, guide cube (500) of the present example includes a post (504), which is configured to receive locking flap (502) as an attachment member. Post (504) includes caps (506) at each end of post (504). Caps (506) are configured to restrict longitudinal movement of locking flap (502) along post (504). While guide cube (500) shown in FIGS. 16, 19, and 23-24 includes two caps (506), it should be understood that post (504) may include only one cap (506) or no caps (506) at all. It should also be understood that a variety of other structures or features may be provided in addition to or in lieu of caps (506). In some versions, post (504) may function as a self-grounding member of guide cube (500) as described above.

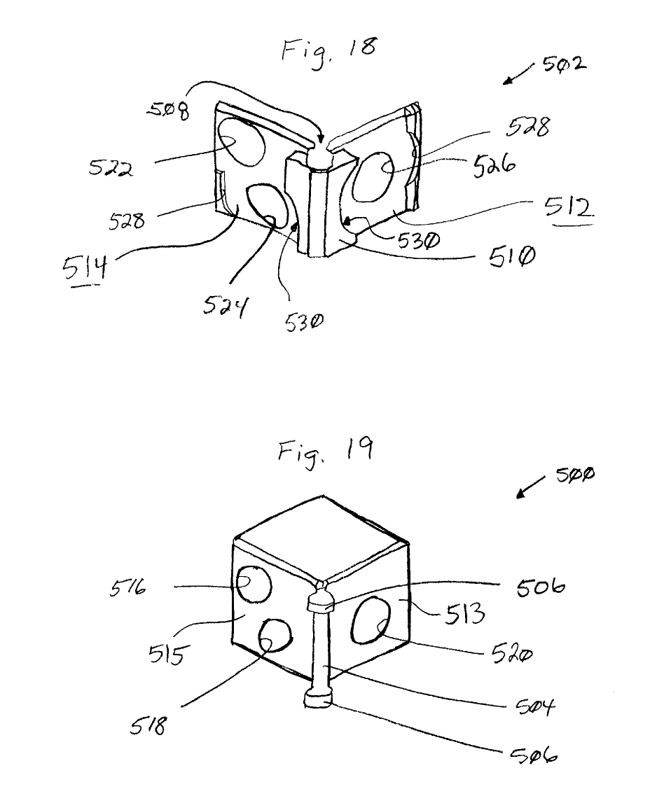

As shown in FIGS. 16 and 18, locking flap (502) of the present example includes a channel (508), which is configured to receive post (504) of guide cube (500) and thereby act as an attachment member. In some versions, locking flap (502) may have a snap-fit to post (504), where post (504) snaps into channel (508). In some other versions, one or both caps (506) of post (504) may be selectively removable such that one cap (506) may be removed, locking flap (502) may be slid into position over post (504), and cap (506) may be replaced on post (504) to engage locking flap (502) to guide cube (500). Various other ways in which locking flap (502) may be coupled with guide cube (500), including but not limited to various other ways in which post (504) and channel (508) may engage as well as various alternatives to post (504) and/or channel (508), will be apparent to those of ordinary skill in the art in view of the teachings herein.

Referring to FIGS. 17, 20, and 21, another exemplary guide cube (600) includes a shaft (604), which is configured to receive locking flap (602) as an attachment member. Shaft (604) includes base member (606) at one end of shaft (604). Base member (606) is configured to restrict longitudinal movement of locking flap (602) along shaft (604). While guide cube (600) shown in FIGS. 17 and 21 includes one base member (606), it should be understood that shaft (604) may alternatively include two base members (606) (e.g., at opposite ends of shaft (604), etc.) or no base members (606) at all. It should also be understood that a variety of other structures or features may be provided in addition to or in lieu of base members (606). In some versions, shaft (604) may function as a self-grounding member of guide cube (600) as described above.

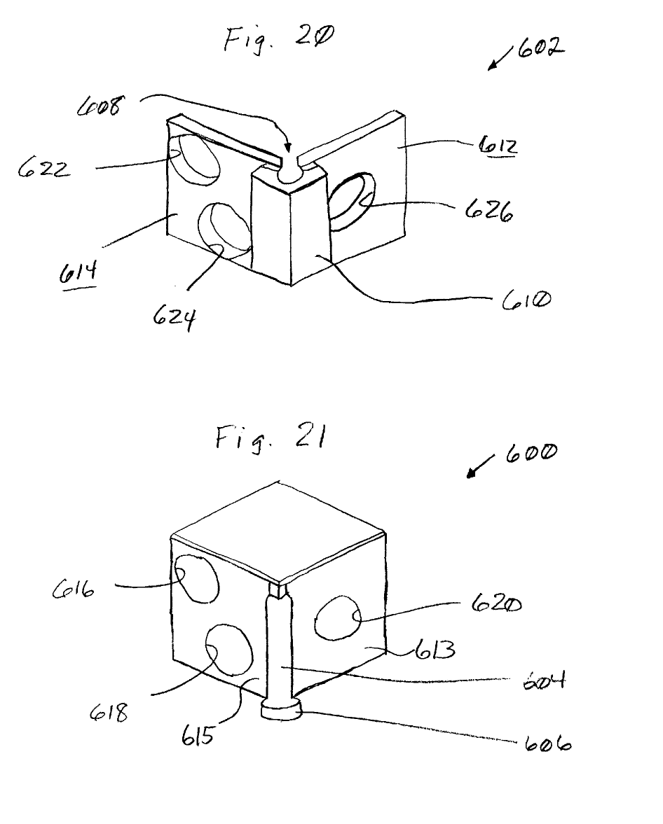

As shown in FIGS. 17 and 20, locking flap (602) of the present example includes a channel (608), which is configured to receive shaft (604) of guide cube (600) and thereby act as an attachment member. In some versions, locking flap (602) may be engaged with guide cube (600) by sliding channel (608) of locking flap (602) over shaft (604) of guide cube (600) until locking flap (602) contacts base member (606). Removal of locking flap (602) from guide cube (600) may be achieved by sliding locking flap (602) in the reverse direction along shaft (604) of guide cube (600). In some other versions, locking flap (602) may have a snap-fit to shaft (604), where shaft (604) snaps into channel (608). Removal of locking flap (602) may be achieved by unsnapping shaft (604) from channel (608) or by sliding channel (608) along shaft (604) until locking flap (602) disengages from guide cube (600). Various other ways in which locking flap (602) may be coupled with guide cube (600), including but not limited to various other ways in which shaft (604) and channel (608) may engage as well as various alternatives to shaft (604) and/or channel (608), will be apparent to those of ordinary skill in the art in view of the teachings herein.

The above modes of engaging locking flaps (502, 602) to guide cubes (500, 600) are merely exemplary. As noted above, various other modes of attaching or otherwise engaging locking flaps (502, 602) to guide cubes (500, 600) will be apparent to those of ordinary skill in the art in view of the teachings herein. By way of further example, a guide cube may be fitted with more than one post (504) or shaft (604) for receiving locking flaps (502, 602). In some such versions, it will be appreciated that locking flaps (502, 602) may be engaged to a guide cube in various positions to accommodate multiple guide holes of the guide cube. In still other versions, post (504) and shaft (604) may be included with locking flaps (502, 602) in lieu of channels (508, 608). Similarly, channels (508, 608) may be included with guide cubes (500, 600) in lieu of post (504) and shaft (604) respectively. As yet another merely illustrative alternative, locking flaps (502, 602) and guide cubes (500, 600) may present a standard hinge configuration, with a separate pin being inserted into coaxially aligned passages formed in locking flaps (502, 602) and guide cubes (500, 600). Further yet, a guide cube (500, 600) may be fitted with multiple channels to receive a post or shaft of a locking flap (502, 602).

In still other versions, locking flaps (502, 602) may be engaged with guide cubes (500, 600) by other means altogether. By way of example only, locking flaps (502, 602) may engage guide cubes (500, 600) by use of an adhesive or other chemical bonding technique. Also by way of example only, locking flaps (502, 602) may engage guide cubes (500, 600) by use of a hook and loop connection or other mechanical fastening technique. As still another merely illustrative variation, a locking flap (502, 602) may be coupled with guide cube (500, 600) via a living hinge. For instance, such a living hinge may be provided by forming locking flap (502, 602) and guide cube (500, 600) integrally together; or by providing a living hinge as an integral part of locking flap (502, 602) or guide cube (500, 600) in then coupling locking flap (502, 602) with guide cube (500, 600) in any suitable fashion.

Referring again to FIGS. 18 and 20, locking flaps (502, 602) of the present example also include rectangular prisms (510, 610), which may act as a grounding means that abut grid plate (96) when guide cubes (500, 600) are inserted in grid plate (96). In other words, rectangular prisms (510, 610) may be configured such that when locking flaps (502, 602) are engaged with guide cubes (500, 600), rectangular prisms (510, 610) prevent over-insertion of guide cubes (500, 600) in grid plate (96). Based on the teachings herein, it will be appreciated that grounding structures may be incorporated on guide cubes (500, 600) alone, on locking flaps (502, 602) alone, or on both guide cubes (500, 600) and locking flaps (502, 602). Of course, a variety of other types of structures, features, or components may be used to provide grounding. Various suitable supplements to and/or substitutes for rectangular prisms (510, 610) will be apparent to those of ordinary skill in the art in view of the teachings herein.

Locking flaps (502, 602) may be constructed from a variety of materials compatible with the imaging techniques described above. In some versions, locking flaps (502, 602) may be made of an elastomeric material. By way of example only, suitable elastomeric materials may include thermosetting plastics that may require vulcanization, thermoplastic elastomers (e.g. Santoprene™ among others), natural rubber, synthetic rubbers (e.g. ethylene propylene diene M-class—EPDM—among others), and other polymers having suitable elastic properties. In some versions, locking flaps (502, 602) may be made of rigid materials that may not compress, but that may permit a degree of flexibility and/or resilience. By way of example only, such suitable materials may include plastic, certain non-magnetic metals, and other suitable polymeric materials. Furthermore, locking flaps (502, 602) may be formed of a combination of resilient non-elastomeric material and elastomeric material. For instance, locking flap (502, 602) may comprise an elastomeric material (e.g., Santoprene) that is applied over a resilient non-elastomeric material (e.g., hard plastic). In the present example, locking flap (502) is formed of a resilient non-elastomeric material (e.g., hard plastic); while locking flap (602) is formed at least in part of an elastomeric material (e.g., Santoprene or some type of rubber). Other suitable materials and combinations of materials will be apparent to those of ordinary skill in the art in view of the teachings herein.

In use, locking flaps (502, 602) may provide a securing mechanism that enables guide cubes (500, 600) to be securely positioned within grid plate (96). Referring to FIGS. 16, 17, 18, and 20, each locking flap (502, 602) may define an obtuse angle. For example, and as shown in FIGS. 16, 18, and 24, locking flap (502) includes first face (512) and second face (514). First and second faces (512, 514) may be associated along one side, thus forming an angle at channel (508). Referring to FIGS. 16 and 24, the angle created by first and second faces (512, 514) of locking flap (502) may be obtuse. It should be understood that adjacent outer faces (513, 515) of guide cube (500) are at right angles relative to each other in the present example. Thus, in some versions, first face (512) and/or second face (514) may not initially contact a corresponding face (513, 515) of guide cube (500) when locking flap (502) is engaged with guide cube (500) and both are located outside of grid plate (96). For instance, as shown in FIG. 24, face (512) of locking flap (502) is angled away from face (513) of guide cube (500); while face (514) of locking flap (502) is in planar contact with face (515) of guide cube (500). In this example, locking flap (502) may be rotated about post (504) such that face (514) of locking flap (502) is in planar contact with face (513) of guide cube (500); and such that face (514) of locking flap (502) is angled away from face (515) of guide cube (500).

When guide cube (500) is fitted with locking flap (502) and is positioned within grid plate (96), one or more of first and second faces (512, 514) may deflect and/or compress against the inner walls of grid plate (96) thereby securing guide cube (500) within grid plate (96). Guide cube (500) with locking flap (502) may thus have a snap fit or interference fit with grid plate (96). In some versions, if guide cube (500) is inserted into grid plate (96) such that first face (512) is disposed in a square recess (130) of grid plate (96), first face (512) is substantially parallel to the adjacent outer face (513) of guide cube (500). However, second face (514) may continue to define an obtuse angle with first face (512) after guide cube (500) is inserted in grid plate (96). That is, second face (514) may be non-parallel with the outer face (515) of guide cube (500) that is adjacent to second face (514) (e.g., such that second face (514) defines an acute angle with the outer face (515) of guide cube (500)). Such a non-parallel orientation of the exposed second face (514) may assist in securing instruments relative to guide cube (500), as will be described in greater detail below. Of course, it should be understood that guide cube (500) may alternatively be inserted into grid plate (96) such that second face (514) is disposed in square recess (130) of grid plate (96). As should be apparent to one of ordinary skill in the art in view of the teachings herein, in such a configuration, second face (514) of locking flap (502) may be substantially parallel with outer face (515) of cube (500); while first face (512) of locking flap (502) may be exposed and non-parallel with outer face (513) of cube (500).

In some versions, locking flap (502) is unitarily rotatable about post (504). In other words, locking flap (502) may be rocked about post (504) such as to selectively move first face (512) of locking flap (502) into contact with outer face (513) of guide cube (500); or to selectively move second face (514) of locking flap (502) into contact with outer face (515) of guide cube (500). In some such versions, as one face (512, 514) of locking flap (502) is rotated into contact with the corresponding outer face (513, 515) of guide cube (500), the other face (514, 512) of locking flap (502) may rotate further away from the other corresponding face (515, 513) of guide cube (500). In other words, as the angle between one face (512, 514) of locking flap (502) and the corresponding outer face (513, 515) of guide cube (500) decreases through rotation of locking flap (502) about post (504), the angle between the other face (514, 512) of locking flap (502) and the other corresponding face (515, 513) of guide cube (500) may increase. In some other versions, locking flap (502) does not unitarily rotate about post (504). For instance, in some such versions, faces (512, 514) may rotate relative to post (504) independent of each other. In other words, in some such versions, movement of one face (512, 514) of locking flap (502) toward or away from the corresponding face (513, 515) of guide cube (500) does not result in any change in the angle defined by the other face (514, 512) of locking flap (502) and the other corresponding face (515, 513) of guide cube (500). Still other suitable ways in which faces (512, 514) of locking flap (502) may move will be apparent to those of ordinary skill in the art in view of the teachings herein.

In some versions, such as those where locking flap (502) is constructed at least in part from an elastomeric material, locking flap (502) may form a right angle such that first and second faces (512, 514) contact corresponding faces (513, 515) of guide cube (500) when locking flap (502) is engaged with guide cube (500) and located outside of grid plate (96). In some such versions, when guide cube (500) is fitted with locking flap (502) and positioned within grid plate (96), one or more of first and second faces (512, 514) may compress against the inner walls of grid plate (96) thereby securing guide cube (500) within grid plate (96).

Similarly, locking flap (602) shown in FIGS. 17 and 20 includes first face (612) and second face (614). First and second faces (612, 614) may be associated along one side, thus forming an angle at channel (608). Referring to FIG. 17, the angle created by first and second faces (612, 614) of locking flap (602) may be obtuse. It should be understood that adjacent outer faces (613, 615) of guide cube (600) are at right angles relative to each other in the present example. Thus, in some versions, first face (612) and/or second face (614) may not initially contact a corresponding face (613, 615) of guide cube (600) when locking flap (602) is engaged with guide cube (600) and both are located outside of grid plate (96).

When guide cube (600) is fitted with locking flap (602) and positioned within grid plate (96), one or more of first and second faces (612, 614) may deflect and/or compress against the inner walls of grid plate (96) thereby securing guide cube (600) within grid plate (96). Guide cube (600) with locking flap (602) may thus have a snap fit or interference fit with grid plate (96). Thus, faces (612, 614) of locking flap (602) may interact with and relative to faces (613, 615) of guide cube (600) in a manner similar to that described above with respect to similar components of locking flap (502) and guide cube (500). Similarly, locking flap (602) may be unitarily rotatable about shaft (604) or may not be unitarily rotatable about shaft (604), as described above with respect to rotatability of locking flap (502). Still other suitable ways in which faces (612, 614) of locking flap (602) may move will be apparent to those of ordinary skill in the art in view of the teachings herein.

In some versions, such as those where locking flap (602) is constructed from an elastomeric material, locking flap (602) may form a right angle such that first and second faces (612, 614) contact corresponding faces (613, 614) of guide cube (600) when locking flap (602) is engaged with guide cube (600) and located outside of grid plate (96). In some such versions, when guide cube (600) is fitted with locking flap (602) and positioned within grid plate (96), one or more of first and second faces (612, 614) may compress against the inner walls of grid plate (96) thereby securing guide cube (600) within grid plate (96).

Based on the teachings herein, it will be appreciated by those of ordinary skill in the art that locking flaps (502, 602) may be configured to have any angled configuration, including oblique, right, or acute angles. It will further be appreciated that locking flaps (502, 602) may fit with guide cubes (500, 600) such that a portion of locking flaps (502, 602) may protrude from guide cube (500, 600). Alternatively or in addition, locking flaps (502, 602) may have a flush fit with guide cubes (500, 600). It will also be appreciated by those of ordinary skill in the art in view of the teachings herein that locking flaps (502, 602) may be designed to fit with guide cubes (500, 600) in grid plate (96); and when positioned within grid plate (96), locking flaps (502, 602) may exert an outward force on at least one of the inner walls of grid plate (96) to secure guide cubes (500, 600) within grid plate (96). It will be appreciated, based on the teachings herein, that such outward force may be accomplished by the deflecting resilient nature of locking flaps (502, 602), by the compressive nature of locking flaps (502, 602), by a combination of deflection resilience and compression, or otherwise.

Furthermore, while a deflecting resilient nature of locking flaps (502, 602) may be related to the properties of the material used for constructing locking flaps (502, 602), the deflection may also be accomplished using other mechanisms. By way of example only, locking flaps (502, 602) could be modified to include a hinged spring mechanism loading faces (512, 514) of locking flap (502) or faces (612, 614) of locking flap (602). In such versions, the force of the spring may bias faces (512, 514, 612, 614) of locking flaps (502, 602) to define an obtuse angle relative to each other and/or to be non-parallel with corresponding faces (513, 515, 613, 615) of guide cubes (500, 600). In some such versions, when locking flaps (502, 602) are engaged with guide cubes (500, 600) and placed within grid plate (96), the inserted face (512, 514, 612, 614) may deflect inward and the spring will be compressed. The compression of the spring (or other type of resilient member) may exert an outward force on the corresponding inner walls of grid plate (96) (e.g., the inner wall engaged by inserted face (512, 514, 612, 614) and the opposite inner wall), thereby increasing friction to further secure guide cube (500, 600) within grid plate (96).

Various other suitable ways to adapt a locking flap (502, 602) to secure a guide cube (500, 600) within a grid plate (96) will be apparent to those of ordinary skill in the art in view of the teachings herein. Furthermore, it will be appreciated by those of ordinary skill in the art in view of the teachings herein that a locking flap (502, 602) may be configured for use with a rotatable guide cube (500, 600), thereby allowing the combined locking flap (502, 602) and guide cube (500, 600) to be rotated such that a variety of guide holes (516, 518, 520, 616, 618, 620) may be used for a procedure. Still in other versions, the guide cube (500, 600) and locking flap (502, 602) may be rotated independently to accommodate a range of guide holes (516, 518, 520, 616, 618, 620) of a guide cube (500, 600). By way of example only, guide cube (500) may be inserted into a selected recess (130) of grid plate (96) with an orientation such that face (512) of locking flap (502) engages any one of the following: the right-hand sidewall interior of the selected recess (130), the left-hand sidewall interior of the selected recess (130), the top sidewall interior of the selected recess (130), or the bottom sidewall interior of the selected recess (130). Alternatively, guide cube (500) may be inserted into a selected recess (130) of grid plate (96) with an orientation such that face (514) of locking flap (502) engages any one of the following: the right-hand sidewall interior of the selected recess (130), the left-hand sidewall interior of the selected recess (130), the top sidewall interior of the selected recess (130), or the bottom sidewall interior of the selected recess (130). Similarly, guide cube (600) may be inserted into a selected recess (130) of grid plate (96) such that face (612) or face (614) of locking flap (602) engages the sidewall interior of any side of the selected recess (130). Still other suitable ways in which a guide cube (500, 600) and/or locking flap (502, 602) may be engaged with a grid plate (96) will be apparent to those of ordinary skill in the art in view of the teachings herein.

In use, locking flaps (502, 602) may further provide a securing mechanism that enables an instrument such as a biopsy device (14) or a portion of a biopsy device (14) (e.g., needle (90) of biopsy device (14), a combination of cannula (94) and obturator (92), etc.) to be securely positioned within a guide hole (516, 518, 520, 616, 618, 620) of guide cube (500, 600). Referring to FIGS. 16, 18, and 19, guide cube (500) of the present example includes guide holes (516, 518, 520), each of which passes completely through guide cube (500). Locking flap (502) of the present example includes corresponding openings or guide holes (522, 524, 526) that may generally align with guide holes (516, 518, 520) respectively. For instance, locking flap (502) may be configured such that guide hole (526) is coaxially aligned with guide hole (520) when first face (512) is against and parallel to face (513) of cube (500); yet such that guide hole (526) is not coaxially aligned with guide hole (520) when first face (512) is angled away from face (513) of cube (500). Similarly, locking flap may be configured such that guide holes (522, 524) are coaxially aligned with corresponding guide holes (516, 518) when second face (514) is against and parallel to face (515) of cube (500); yet such that guide holes (522, 524) are not coaxially aligned with corresponding guide holes (516, 518) when second face (514) is angled away from face (515) of cube (500). It should be understood that, in some versions, and as described above, one face (512, 514) of locking flap (502) may be angled away from the corresponding face (513, 515) of guide cube (500) when such face (512, 514) is “exposed” from grid plate (96) (e.g., while the other face (514, 512) of locking flap (502) is inserted with the guide cube (500) in grid plate (96)).

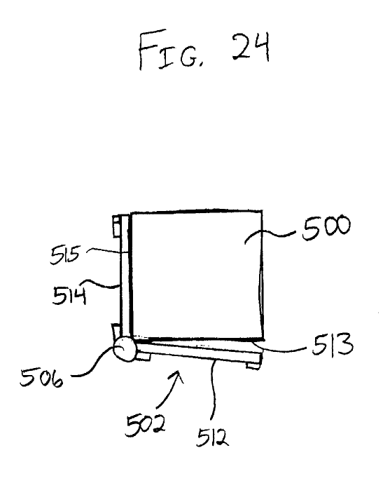

FIGS. 23-24 illustrate an example of face (512) of locking flap (502) being angled away from the corresponding face (513) of guide cube (500), such as when face (512) is “exposed” relative to grid plate (96) (not shown in FIGS. 23-24) while face (514) is inserted with guide cube (500) in grid plate (96). As shown, the axis of guide hole (526) is not aligned with the axis of guide hole (520). In particular, the axis of guide hole (526) is not even parallel with the axis of guide hole (520) in this example. Guide holes (526, 520) thus extend along non-parallel planes. While guide holes (526, 520) have substantially the same diameter in this example, the non-parallel arrangement of guide holes (526, 520) require the planes of guide holes (526, 520) to be substantially parallel in order for an instrument such as a biopsy device (14) or a portion of a biopsy device (14) (e.g., needle (90) of biopsy device (14), a combination of cannula (94) and obturator (92), etc.) to be inserted through both guide holes (526, 520). Since locking flap (502) is resilient in this example, face (512) can be manually bent by the user to make the planes of guide holes (526, 520) substantially parallel enough for an instrument to be inserted through both guide holes (526, 520). For instance, a user may use a finger or a device to manually push face (512) of locking flap (502) toward face (513) of guide cube (500) until face (512) of locking flap (502) contacts face (513) of guide cube (500). With face (512) being bent to make the planes of guide holes (526, 520) substantially parallel enough, thereby bringing the axes of guide holes (526, 520) into substantial alignment, the user may then insert the instrument through guide holes (526, 520). Of course, the bending of face (512) to allow the instrument to be inserted through both guide holes (526, 520) simultaneously may be accomplished using the instrument itself, such as by first inserting the tip of the instrument through guide hole (526) and then into guide hole (520), with the shaft of the instrument essentially forcing face (512) toward face (513) of guide cube (500) during insertion.

Continuing with the above example, with the instrument being inserted through both guide holes (526, 520), the resilience of locking flap (502) will urge face (512) of locking flap (502) toward its initial position, angled away from the corresponding face (513) of guide cube (500). However, the instrument that is inserted through guide holes (526, 520) will prevent face (512) from reaching that initial position. Face (512) will therefore resiliently bear against the instrument that is inserted through guide holes (526, 520). In particular, the edge around guide hole (526) will bear against the instrument that is inserted through guide holes (526, 520). This resilient engagement of locking flap (502) with the instrument that is inserted through guide holes (526, 520) will create friction, providing resistance to proximal movement of the instrument that is inserted through guide holes (526, 520). In other words, the resilient engagement of locking flap (502) with the instrument that is inserted through guide holes (526, 520) will resist withdrawal of the instrument from guide cube (500) in this example. When the user is ready to withdraw the instrument from guide cube (500) the user may manually push on face (512) of guide cube (500), to bring the planes of guide holes (520, 526) back into a substantially parallel relationship (e.g., bringing the guide holes (520, 526) back into a substantially coaxial relationship). With face (512) being in such a position (e.g., substantially parallel to face (513) of guide cube (500)), the instrument may be withdrawn from guide cube (500) with relative ease. Alternatively, the instrument and guide cube (500) may be removed from grid plate (96) together, and then guide cube (500) may be later removed from the instrument.

In some versions, openings or guide holes (522, 524, 526) of locking flap (502) may be undersized compared to guide holes (516, 518, 520) of guide cube (500) (and compared to the outer diameter of the instrument that is to be inserted through guide cube (500)). In some such versions, particularly where locking flap (502) is comprised of an elastomeric material, when an instrument is inserted within guide cube (500), openings or guides holes (522, 524, 526) of locking flap (502) will deflect and/or expand around the instrument and permit the instrument to pass into guide holes (516, 518, 520) of guide cube (500). The undersized nature of locking flap (502) openings or guide holes (522, 524, 526) may thus create a compressive force around the instrument, thereby substantially securing the instrument relative to the guide cube (500). In some versions, the securing force provided by locking flap (502) is sufficient to secure an instrument against undesired or inadvertent proximal displacement when the instrument contacts tissue during a biopsy or other procedure.

Similarly, referring to FIGS. 17, 20, and 21, guide cube (600) of the present example includes guide holes (616, 618, 620). Locking flap (602) of the present example includes corresponding openings or guide holes (622, 624, 626) that may generally align with guide holes (616, 618, 620) respectively. In some versions, particularly where locking flap (602) is formed at least in part of a resilient yet somewhat rigid material, locking flap (602) may resiliently bear against an instrument that is inserted through a guide hole (622, 624, 626) of locking flap (602) and a corresponding guide hole (616, 618, 620) of guide cube (600). Such resilient bearing may provide substantial resistance to axial withdrawal of the instrument from guide cube (600), in a manner as described in detail above with reference to FIG. 23 in the context of guide cube (500).

In some versions, openings or guide holes (622, 624, 626) of locking flap (602) may be undersized compared to guide holes (616, 618, 620) of guide cube (600). In some such versions, particularly where locking flap (602) is comprised of an elastomeric material, when an instrument is inserted within guide cube (600), openings or guides holes (622, 624, 626) of locking flap (602) will deflect and/or expand around the instrument and permit the instrument to pass into guide holes (616, 618, 620) of guide cube (600). The undersized nature of locking flap (602) openings or guide holes (622, 624, 626) may thus create a compressive force around the instrument, thereby substantially securing the instrument relative to the guide cube (600). In some versions, the securing force provided by locking flap (602) is sufficient to secure an instrument against undesired or inadvertent proximal displacement when the instrument contacts tissue during a biopsy or other procedure.

In some versions, locking flaps (502, 602) may be constructed without clearly defined openings or guide holes (522, 524, 526, 622, 624, 626), and instead locking flaps (502, 602) may be made from a penetrable material such that openings or guide holes may be created in locking flaps (502, 602) by the act of inserting an instrument. In some such versions, the penetrable material may compress around the inserted instrument thereby further securing the instrument within a corresponding guide hole (516, 518, 520, 616, 618, 620) of guide cube (500, 600). Based on the teachings herein, it will also be appreciated that locking flaps (502, 602) may include expandable slits or star-shaped openings in lieu of defined openings or guide holes (522, 524, 526, 622, 624, 626). Still other suitable ways of accommodating insertion of an instrument through locking flaps (502, 602) will be apparent to those of ordinary skill in the art in view of the teachings herein.

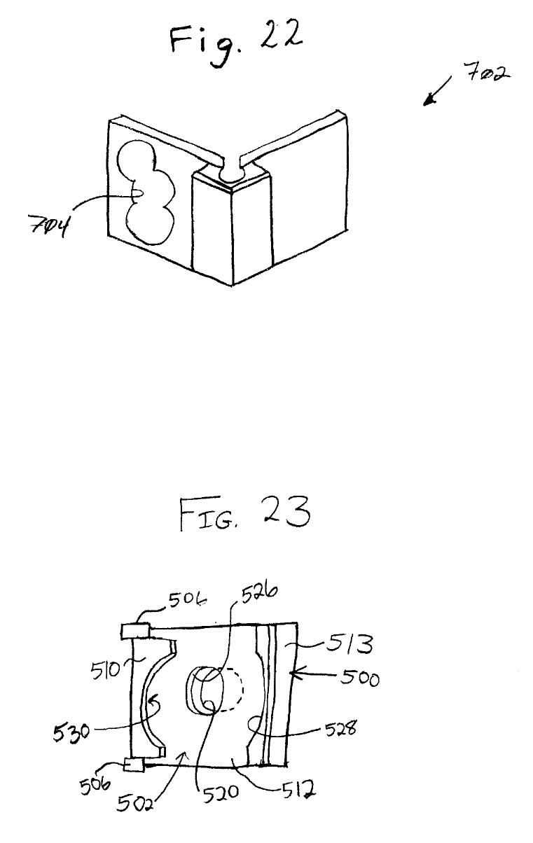

Still in some other versions, as shown in FIG. 22, locking flap (702) may include overlapping openings or guide holes (704) that may accommodate a rotatable guide cube capable of having guide holes in various locations. Based on the teachings herein, it will be appreciated that a 2-axis or 3-axis rotatable guide cube and rotatable locking flap may be configured for adjustable engagement such that numerous guide hole positions of a guide cube may be accommodated by locking flap. By way of example only, guide cube (104) of FIG. 8 may be adapted to work with a rotatable locking flap such that the guide hole positions shown in FIG. 9 may be accommodated with a single guide cube and locking flap adjustable combination.

Referring now to FIGS. 16 and 18, another feature that may be incorporated into locking flap (502) may include radial recesses (528, 530). Such radial recesses (528, 530) may be formed in rectangular prism (510) and/or along other portions of locking flap (502) and may allow grounding of an instrument, such as biopsy device (14) or a portion of biopsy device (14) (e.g. depth stop device (95)), against first and/or second faces (512, 514). In other words, recesses (528, 530) may provide clearance for a portion of an instrument that is inserted in guide cube (500), allowing the instrument to be more fully inserted in a guide hole (516, 518, 520) of guide cube (500). In some versions, radial recesses (528, 530) may also assist in preventing an instrument, such as a biopsy device (14) or a portion of a biopsy device (14), from angulation during use; or at least reducing the amount of angulation.

In some versions, where locking flaps (502, 602) are constructed of non-compressible materials, an instrument may be securely held within a guide cube (500, 600) by using other features. By way of example only, the o-ring grooves and inserts or elastomeric rings described in U.S. Patent Application Ser. No. [Attorney Docket No. END6621.0567779], entitled “MRI Biopsy Targeting Cube with Eccentric Lock,” filed ______, the disclosure of which is incorporated by reference herein, may be adapted for use with guide cubes (500, 600) and/or locking flaps (502, 602) to assist in securing an instrument within guide cubes (500, 600). Still other various ways to suitably secure an instrument within a guide hole (516, 518, 520, 616, 618, 620) of a guide cube (500, 600) that incorporates a locking flap (502, 602) of non-compressible construction or compressible construction will be apparent to those of ordinary skill in the art in view of the teachings herein. It should also be understood that any guide cube (104, 104a, 104b, 104c, 500, 600) described herein may be modified to include a locking flap (502, 602) as described herein or any suitable variation of a locking flap (502, 602) described herein. In other words, guide cubes (500, 600) are not necessarily the only types of guide cubes that may incorporate a locking flap (502, 602).

While locking flap (502) in the foregoing examples has two faces (512, 514), it should be understood that flap (502) may alternatively have just one face (512). For instance, such a face (512) may be resiliently biased to be angled away from a corresponding face (513) of guide cube (500) as described above in the context of a two-faced locking flap (502). Locking flap (602) may also have similar variations. In addition, while locking flap (502) in the foregoing examples is a substantially unitary construction, and to the extent that a locking flap (502) has two faces (512, 514), it should be understood that faces (512, 514) may be formed separately and/or joined to guide cube (500) separately. Again, locking flap (602) may also have similar variations. Furthermore, in some versions, a given face (512, 514, 612, 614) may simply lack any guide holes (522, 524, 526, 622, 624, 626) altogether. Still other suitable variations of locking flaps (502, 602) and/or guide cubes (500, 600) will be apparent to those of ordinary skill in the art in view of the teachings herein.

The guide cubes (104, 104a, 104b, 104c, 500, 600) and locking flaps (502, 602) described herein, or components thereof, may be made using a molding process, an extrusion process, or any other suitable manufacturing process. By way of example only, and not limitation, other suitable manufacturing processes may provide that some components may be stamped from non-magnetic metals. Furthermore, the guide cubes (104, 104a, 104b, 104c, 500, 600) and locking flaps (502, 602) may be constructed as distinct components or as single molded components. Where components are molded, single material constructions may be used or multiple material constructions may be achieved by over-molding processes. Still other suitable manufacturing processes and techniques to construct the various guide cubes (104, 104a, 104b, 104c, 500, 600) and locking flaps (502, 602) described herein will be apparent to those of ordinary skill in the art in view of the teachings herein.

As noted above, any guide cube (104, 104a, 104b, 104c, 500, 600) described herein may be used in a procedure that includes the use of PEM imaging, BSGI imaging, or any other suitable type of imaging. By way of example only, a guide cube (104, 104a, 104b, 104c, 500, 600) may be used with a grid plate (96) that is configured for use in an MRI setting, a grid plate for use in a nuclear/molecular imaging setting, or with some other type of cube holder (e.g., “guide holder”) used in nuclear/molecular imaging or other type of imaging. For instance, a suitable alternative cube holder or “guide holder” may include fewer openings (e.g., one to four) that are configured to receive a guide cube (104, 104a, 104b, 104c, 500, 600) as compared to the number of recesses (130) in grid plate (96). Furthermore, a guide cube (104, 104a, 104b, 104c, 500, 600) may be used with a biopsy device (14) in conjunction with a full targeting set or with just a biopsy device (14) (e.g., in settings where a radioisotope can be communicated through the biopsy device (14)). It should also be understood that a guide cube (104, 104a, 104b, 104c, 500, 600) may be used just with a radioisotope, without necessarily involving any biopsy device (14). For instance, a radioisotope may be provided on or through an implement that has a sharp tip, and the implement may be inserted through the guide cube (104, 104a, 104b, 104c, 500, 600). Still other various settings and combinations in which a guide cube (104, 104a, 104b, 104c, 500, 600) may be used will be apparent to those of ordinary skill in the art in view of the teachings herein.

While several guide cubes have been discussed in detail above, it should be understood that the components, features, configurations, and methods of using the guide cubes discussed are not limited to the contexts provided above. In particular, components, features, configurations, and methods of use described in the context of one of the guide cubes may be incorporated into any of the other guide cubes. One merely exemplary additional feature that may be provided in any of the guide cubes described herein is one or more ridges on one or more external faces of the cube. Such ridges may be substantially rigid, elastomeric, or have any other suitable properties. Such ridges may provide a more secure fit between a cube and grid (e.g., reducing the likelihood that that the guide cube will undesirably fall out of the grid plate), may permit a single cube to be inserted in different grids having differently sized openings, and/or may provide other results. Still other additional and alternative suitable components, features, configurations, and methods of using the guide cubes will be apparent to those of ordinary skill in the art in view of the teachings herein.

Versions of the present invention have application in conventional endoscopic and open surgical instrumentation as well as application in robotic-assisted surgery.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, embodiments of the device may be disassembled, and any number of the particular pieces or parts of the device may be selectively replaced or removed in any combination. Upon cleaning and/or replacement of particular parts, embodiments of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various versions in the present disclosure, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

What is claimed is:

1. A guide device for guiding a medical instrument relative to a patient, the guide device being usable with a first plate and a second plate, wherein the first plate has a frame defining a plurality of apertures, wherein the second plate and the first plate are adjustable to secure a portion of the patient, wherein the guide device is configured to be coupled with a selected one of the apertures of the first plate, the guide device comprising:
a. a body having a first side and a second side, wherein the first side is opposite from the second side, the body further defining a first passageway extending from an opening formed in the first side to an opening formed in the second side; and
b. a first flap member, wherein the first flap member is coupled with the body, wherein the first flap member is positioned adjacent to the first side, wherein the first flap member is pivotable relative to the first side, wherein the first flap member defines an opening configured to substantially align with the opening formed in the first side;
wherein the opening of the first flap member and the first passageway of the body are configured to receive at least a portion of the medical instrument.

2. The guide device of claim 1, wherein the body further comprises a third side and a fourth side, wherein the third side is opposite from the second side, wherein the third side is adjacent to both the first side and the second side, wherein the fourth side is adjacent to both the first side and the second side.

3. The guide device of claim 2, the body further defining a second passageway extending from an opening formed in the third side to an opening formed in the fourth side.

4. The guide device of claim 3, further comprising a second flap member, wherein the second flap member is coupled with the body.

5. The guide device of claim 4, wherein the second flap member is positioned adjacent to the third side, wherein the second flap member is pivotable relative to the third side.

6. The guide device of claim 5, wherein the second flap member defines an opening configured to substantially align with the opening formed in the third side.

7. The guide device of claim 5, wherein the first side of the body and the third side of the body together define a right angle.

8. The guide device of claim 7, wherein the first flap member and the second flap member together define an obtuse angle.

9. The guide device of claim 8, wherein the first flap member and the second flap member are configured such that the first flap member pivots away from the first side of the body as the second flap member pivots toward the third side of the body.

10. The guide device of claim 1, wherein the first flap member is resiliently biased to define a non-parallel angle relative to the first side of the body.

11. The guide device of claim 10, wherein the opening of the first flap member is centered on a first axis, wherein the passageway of the body defines a second axis, wherein the first axis is non-parallel to the second axis when the first flap member defines a non-parallel angle relative to the first side of the body.

12. The guide device of claim 1, wherein the first flap member is coupled with the body by a snap fit connection or a sliding connection.

13. The guide device of claim 1, wherein the first flap member is formed of an elastomeric material.

14. The guide device of claim 13, wherein the elastomeric material is operatively configured to compress when the body and the first flap member are inserted within the selected one of the plurality of apertures of the first plate.

15. The guide device of claim 1, wherein the body further includes a plurality of passageways extending from associated openings formed in the first side to associated openings formed in the second side.

16. The biopsy device of claim 15, wherein the first flap member defines a plurality of openings configured to substantially align with corresponding openings formed in the first side.

17. The biopsy device of claim 1, wherein the first flap member is configured to resiliently bear against an instrument that is inserted through the opening of the first flap member and the first passageway of the body to resist withdrawal of the instrument from the body.

18. A guide device for guiding a medical instrument relative to a patient, the guide device being usable with a first plate and a second plate, wherein the first plate has a frame defining a plurality of apertures, wherein the second plate and the first plate are adjustable to secure a portion of the patient, wherein the guide device is configured to be coupled with a selected one of the apertures of the first plate, the guide device comprising:
a. a body having a first side, a second side, a third side, and a fourth side, wherein the first side is opposite from the second side, wherein the third side is opposite from the fourth side, wherein the third side is adjacent to both the first side and the second side, wherein the fourth side is adjacent to both the first side and the second side, the body further defining a first passageway extending from an opening formed in the first side to an opening formed in the second side and a second passageway extending from an opening formed in the third side to an opening formed in the fourth side;
b. a first flap member, wherein the first flap member is coupled with the body, wherein the first flap member is positioned adjacent to the first side, wherein the first flap member is pivotable relative to the first side, wherein the first flap member defines an opening configured to substantially align with the opening formed in the first side; and
c. a second flap member, wherein the second flap member is coupled with the body, wherein the second flap member is positioned adjacent to the third side, wherein the second flap member is pivotable relative to the third side, wherein the second flap member defines an opening configured to substantially align with the opening formed in the third side.

19. The guide device of claim 17, wherein the first side and the third side together define a right angle, wherein the first flap member and the second flap member are resiliently biased to together define an obtuse angle.

20. A guide device for guiding a medical instrument relative to a patient, the guide device being usable with a first plate and a second plate, wherein the first plate has a frame defining a plurality of apertures, wherein the second plate and the first plate are adjustable to secure a portion of the patient, wherein the guide device is configured to be coupled with a selected one of the apertures of the first plate, the guide device comprising:
a. a body having a first side, a second side, a third side, and a fourth side, wherein the first side is opposite from the second side, wherein the third side is opposite from the fourth side, wherein the third side is adjacent to both the first side and the second side, wherein the fourth side is adjacent to both the first side and the second side, the body further defining a first passageway extending from an opening formed in the first side to an opening formed in the second side and a second passageway extending from an opening formed in the third side to an opening formed in the fourth side; and
b. a flap lock coupled with the body along an edge between the first side of the body and the second side of the body, the flap lock comprising:
i. a first flap member positioned over the first side of the body, wherein the first flap member defines an opening configured to substantially align with the opening formed in the first side, and
ii. a second flap member positioned over the third side of the body, wherein the second flap member defines an opening configured to substantially align with the opening formed in the third side.
